(19) United States
(12) Patent Application Publication
(10) Pub. No.: US 2025/0022173 A1
(43) Pub. Date: Jan. 16, 2025

(54) SYSTEM AND METHOD FOR CALIBRATING SYSTEM PARAMETERS FOR IMAGE RECONSTRUCTION

(71) Applicant: Siemens Healthcare Diagnostics Inc., Tarrytown, NY (US)

(72) Inventors: Mohiudeen Azhar, Bangalore (IN); Abhijeet A. Joshi, Bangalore (IN)

(21) Appl. No.: 18/707,875

(22) PCT Filed: Oct. 31, 2022
(86) PCT No.: PCT/IB2022/060454

§ 371 Date: May 6, 2024

Related U.S. Application Data
(60) Provisional application No. 63/276,817, filed on Nov. 8, 2021.

(51) Int. Cl. G06T 7/80; G06T 3/40; G06V 10/141

(52) U.S. Cl. CPC G06T 7/80; G06T 3/40; G06V 10/141; G06T 2207/10056

(57) ABSTRACT

A computer implemented method, an imaging device, an image reconstruction system and a computer program product, for calibrating system parameters of the image reconstruction system are provided, and include obtaining low resolution captured images by illuminating a sample with light source(s), obtaining a high resolution representation of the captured images based on the system parameters and the captured images, generating an approximate function based on the high resolution representation and the captured images, wherein the approximate function is a function of system parameters, and generating an updated set of system parameters by optimizing the approximate function. The computer implemented method, the imaging device, the image reconstruction system and the computer program product enhance speed of calibration of the image reconstruction system by orders of magnitude, thereby, enabling noise and artifact free microscopic imaging in a robust way for the lifetime of the microscope.

### FIELD OF TECHNOLOGY

The present disclosure refers to a method and a system for processing an image. More particularly, the present disclosure refers to a method and a system for processing a medical image. Furthermore, the present disclosure refers to a method and a system for calibrating an image reconstruction system so as to enhance the image quality of the reconstructed image.

### BACKGROUND

High resolution microscopy is an indispensable tool for technicians and/or clinicians working in a pathology to analyze patient samples such as blood, urine sediments, tissue pathology samples, etc. Fourier ptychography microscopy (FPM) is a microscopy technique used for enhancing resolution of an image without compromising the field of view. FPM enables obtaining high resolution images with multiple low resolution images with wide field of view, at varying angles of illumination. The FPM achieves a high resolution image output by illuminating the subject, that is, the sample to be tested, at multiple angles and subsequently stitching the information acquired during each illumination in Fourier domain. To create the multiple illumination angles, light sources arranged in a grid pattern are typically used. Due to its high space-bandwidth imaging capability FPM may be employed in bio-medical imaging applications such as, Haematology, Pathology, Urine analysis etc.

FIG. 1 illustrates a system 100 for generating high resolution representation, that is, a high resolution image 104 from low resolution images 101 using FPM based image reconstruction system 103 employing an image reconstruction algorithm, according to state of the art. The low resolution images 101 are captured by multiple light sources (not shown) such as light emitting diodes (LEDs) illuminating the subject, such as a sample, being illuminated at multiple angles. These raw low resolution images 101 are then provided as an input to the FPM based image reconstruction system 103 employing a Fourier based image reconstruction algorithm for stitching the low resolution images 101 together.

The FPM based image reconstruction system 103 also receives another input in form of system parameters 102. The system parameters 102 include, for example, optical, geo-metrical and/or alignment data such as global LED position misalignments, LED plane to sample separation, magnification, wavelength, thresholding parameters etc. For achieving quality reconstruction, that is, quality high resolution images 104, it is desired to have precise values of the system parameters. Often, system parameters are not known accurately and thereby produce suboptimal image reconstruction. Moreover, such precision is not available through a mere system characterization, and thus a robust calibration technique is required. Also, imaging device such as a Fourier ptychography microscope employed by the image reconstruction system 103 may require periodic calibration and system validation to account for and identify mechanical perturbations or component degradations, if any, thereby highlighting the need for automated calibration procedure.

Conventional methods available for reconstruction of medical images mostly include post processing of medical images on a standard microscope. Moreover, advanced sampling-based methods such as surrogate optimization, genetic algorithms, simulated annealing may be used for calibration. However, these methods tend to optimize number of sampling points, wherein each sampling point constitutes an FPM based image reconstruction. Thus, the actual number of FPM reconstructions required is quite large. Furthermore, each FPM reconstruction is time and resource consuming operation amounting to at least few minutes per reconstruction, making direct implementation of the above algorithms inefficient.

Some conventional methods propose LED position calibration, including individual position correction, however, in none of these methods a general multi-parameter approach is considered for the global parameters.

The object of the present disclosure is therefore to provide a method and a system that enables effective, fast and accurate calibrating of system parameters associated with an image reconstruction system and method, thereby enhancing quality of the reconstructed image(s).

### SUMMARY

The disclosure achieves the object by a computer implemented method for calibrating system parameters associated with an image reconstruction system such that the speed of calibration of the image reconstruction system is increased by orders of magnitude which in turn enhances quality of image reconstruction. The system parameters include, for example, optical data, geometrical data, alignment data, illumination plane to sample separation data, magnification data, wavelength data, and/or thresholding data.

The method comprises obtaining multiple captured images, that is an image of a sample, by illuminating the sample with light source(s) associated with an imaging device of the image reconstruction system. The imaging device may include, for example, a Fourier Ptychography Microscope. Such an imaging device may include, for example, controllable light source(s) placed at discrete positions, a tube lens, one or more objective lenses and an image capturing unit. The light sources may be configured to emit light of a predefined wavelength distribution at predefined angles such that the sample is illuminated at multiple angles. The sample may include any object that may require a magnified visualization. For example, in medical applications, this sample may include a pregnancy test strip, a urine test strip, etc.

Upon obtaining the captured images, the method iteratively performs below steps including obtaining a high resolution representation of the captured images, wherein the high resolution representation is generated by the image reconstruction system based on the system parameters and the captured images. The image reconstruction system processes the captured images and synthesizes the information thus obtained into a final representation of the sample, for example in Fourier domain. The final representation being of a high resolution.

The method uses, for example, a surrogate function such as a forward imaging function that takes the amplitude and phase information of the high resolution representation instead of the actual captured images to generate a sample transmission field that can be represented by the equation given below:

S(r)=Ae^{i(Ø)}

Where S(r) is the sample transmission field, A is the amplitude of the high resolution representation and Ø is the phase of the high resolution representation.

The method thereby generates a sample spectrum S(k) and internally calls the forward imaging function along with system parameter(s). The sample spectrum S(k) can be represented by the equation given below:

S(k)=Recon(I_{captured},P)

Where S(k) represents the high resolution representation, ‘Recon’ represent the reconstruction function, I_{captured} represents the captured images of low resolution and P represents the system parameters.

The iterative steps performed by the method include generating an approximate function based on the high resolution representation and the captured images. The approximate function is a function of system parameters and can be represented by the equation given below:

dₐₚₚ(P)=d[(Simulate(S(k)_{evaluated at P}_{ᵢ},P),I_{captured}]

Where dₐₚₚ represents an approximated distance metric being computed for the system parameter(s) P and Pᵢ represents system parameters from i^{th} iteration performed by method.

The iterative steps performed by the method include generating an updated set of system parameters by optimizing the approximate function. For optimizing the approximate function, the method evaluates the approximate function at multiple sample points in a parameter space of the system parameters, obtains a minimal value of the approximate function for the system parameters, for example, by using various methods of optimization such as genetic algorithms, surrogate optimization techniques, etc., and updates the system parameters corresponding to the minimal value.

For evaluating the approximate function at a particular instance of the system parameters, the method generates simulated images by simulating the captured images based on the high resolution representation generated in a previous iteration of calibration of system parameters, and the particular instance of the system parameters, and obtains a distance metric between the simulated images and the captured images. The simulated images and the captured images are low resolution images. According to an embodiment, the method generates the simulated images by simulating the captured image using sample points in a multi-dimensional parameter space of the system parameters and the amplitude and phase information of the high resolution representation. Advantageously, the method performs calibration for all system parameters at once to achieve multi-parameter optimization.

The simulated images can be represented by the equation given below:

I_{simulated}=Simulate(S(k),P)

Where ‘Simulate’ represents, for example, the forward imaging function used for simulating the captured images using the system parameters. Advantageously, P that is used to generate I_{simulated} can be perturbed around the system parameters used to generate S(k), thereby allowing ample sample point evaluations at a faster rate as Simulate function is orders of magnitude faster to evaluate compared to Recon function. Thus, the optimization of the system parameters can be facilitated by optimization of the approximate function.

After obtaining the distance metric, that is, the approximate function, the method then uses this value that is a function of the system parameters to determine the updated system parameter that can be represented by the equation given below:

Pᵢ₊₁=argminₚ[dₐₚₚ(P)]

Where Pᵢ₊₁ is the system parameter at iteration ‘i+1’ that is relying on the amplitude and phase information, that is used in S(k), from the previous iteration ‘i’ of the method and argmin represents minimal value.

Advantageously, the method proposed herein requires lesser number of iterations of time consuming image reconstruction process compared to calibration techniques known in state of the art.

Advantageously, the method proposed herein feeds the updated system parameter(s) after the last iteration, to the image reconstruction system thereby, enabling speedy calibration of the system parameters which in turn helps in enhancement of the image quality being produced by the image reconstruction system. The enhanced image quality is indicated by sufficient resolution and minimal noise and artifacts.

The object of the disclosure is also achieved by an imaging device of an image reconstruction system. The imaging device comprises an imaging module that illuminates a sample with light source(s) of the imaging device and captures a plurality of images of the sample, that is, multiple low resolution captured images. The imaging device also includes processing unit(s), and a memory coupled to the processing unit(s). The memory comprises a surrogate calibration module configured to perform the method steps as described above.

The object of the disclosure is also achieved by an image reconstruction system. According to an embodiment, the image reconstruction system includes server(s) and an imaging device coupled to the server(s). The server(s) comprise instructions, which when executed causes the server(s) to perform the method steps as described above.

The object of the disclosure is also achieved by a computer program product comprising a computer program, the computer program being loadable into a storage unit of the image reconstruction system or any other system, and including program code sections to make the system execute the method steps described above when the computer program is executed in the system. The disclosure relates in one aspect to a computer-readable medium, on which program code sections of a computer program are saved, the program code sections being loadable into and/or executable in a system to make the system execute the method according to an aspect of the disclosure when the program code sections are executed in the system. The realization of the disclosure by a computer program product and/or a computer-readable medium has the advantage that already existing management systems can be easily adopted by software updates in order to work as proposed by the disclosure. The computer program product can be, for example, a computer program or comprise another element apart from the computer program. This other element can be hardware, for example a memory device, on which the computer program is stored, a hardware key for using the computer program and the like, and/or software, for example a documentation or a software key for using the computer program.

### BRIEF DESCRIPTION OF THE DRAWINGS

The present disclosure is further described hereinafter with reference to illustrated embodiments shown in the accompanying drawings, in which:

FIG. 1 illustrates a system for generating a high resolution representation from low resolution images using FPM based image reconstruction system, according to state of the art;

FIG. 2 illustrates an image reconstruction system generating a reconstructed image of an enhanced quality by employing a surrogate calibration module, according to an embodiment of the present disclosure;

FIG. 3 provides an illustration of a block diagram of a client-server architecture that is a geometric modelling of components representing different parts of real-world objects, according to an embodiment of the present disclosure;

FIG. 4 is a block diagram illustrating an architecture of a computer system employed by the image reconstruction system shown in FIG. 2, according to an embodiment of the present disclosure; and

FIGS. 5A-5B illustrate flowcharts of a computer-implemented method for calibrating system parameters associated with the image reconstruction system shown in FIG. 2, according to an embodiment of the disclosure.

### DETAILED DESCRIPTION

Hereinafter, embodiments for carrying out the present disclosure are described in detail. The various embodiments are described with reference to the drawings, wherein like reference numerals are used to refer to like elements throughout. In the following description, for purpose of explanation, numerous specific details are set forth in order to provide a thorough understanding of one or more embodiments. It may be evident that such embodiments may be practiced without these specific details.

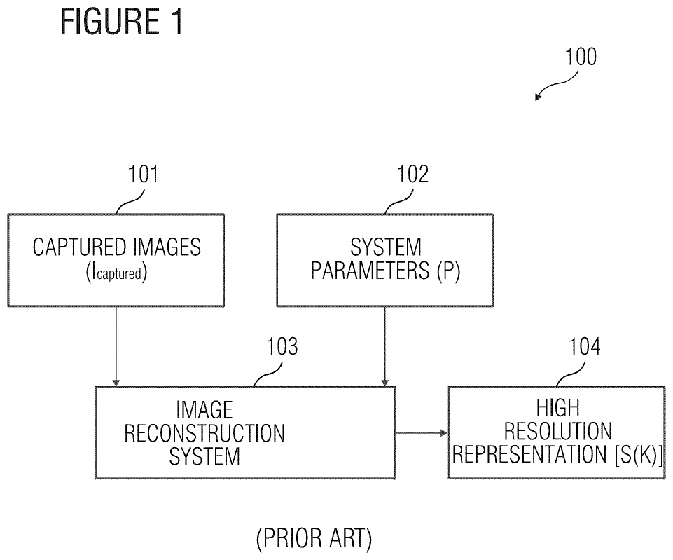

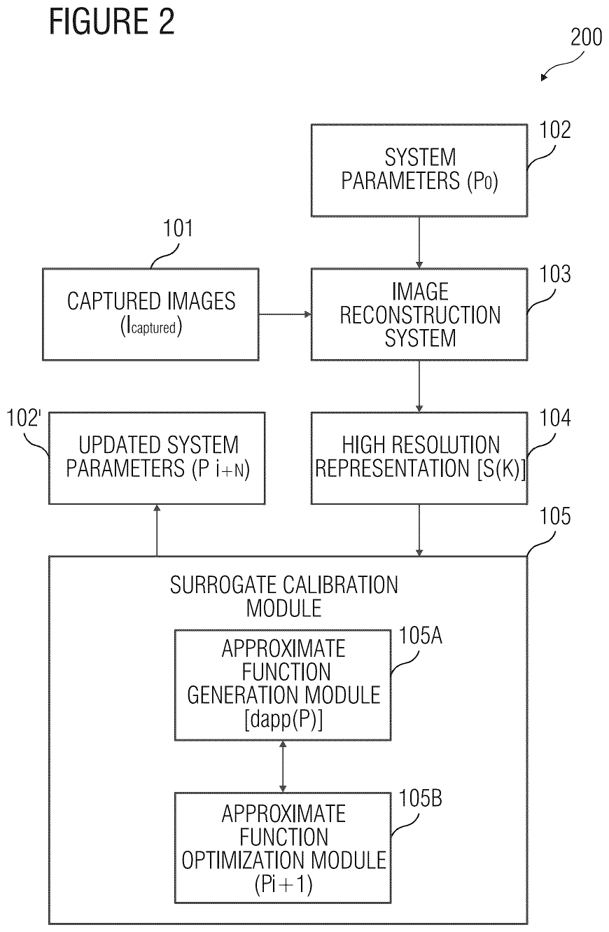

FIG. 2 illustrates an image reconstruction system 103 generating a reconstructed image of an enhanced quality by employing a surrogate calibration module 105, according to an embodiment of the present disclosure. The image reconstruction system 103 and the surrogate calibration module 105 form a part of an image analysis system 200, according to an embodiment of the present disclosure when compared to the image analysis system 100 shown in FIG. 1 according to state of the art. Advantageously, the surrogate calibration module 105 is employed selectively by the image analysis system 100 as and when the calibration of system parameters is to be performed.

The image reconstruction system 103 includes server(s) and an imaging device (not shown) coupled to the server(s). The image reconstruction system 103 employs a surrogate calibration module 105 wherein the server(s) store instructions therein defined by the surrogate calibration module 105 which when executed, cause the servers to obtain a captured image 101 of a low resolution captured by the imaging device by illuminating a sample with light source(s) associated with the imaging device. The image reconstruction system 103 obtains, that is, generates a high resolution representation 104 of the captured images 101 based on system parameters 102 including for example, optical data, geometrical data, alignment data, illumination plane to sample separation data, magnification data, wavelength data, and thresholding data associated with the light source(s) and/or the imaging device used for illuminating a sample and capturing the captured images 101 of the sample.

The high resolution representation S(k) can be represented by the equation given below:

S(k)=Recon(I_{captured},P)

where S(k) represents the high resolution representation, ‘Recon’ represent the reconstruction function, I_{captured} represents the captured images of low resolution and P represents the system parameters.

The image reconstruction system 103 then provides this high resolution representation 104 to the surrogate calibration module 105. A person skilled in the art may appreciate that the surrogate calibration module 105 may reside within or outside the image reconstruction system 103, for example, in a cloud computing environment and being offered in form of software as a service, or as an edge device or solution available for deployment on demand, or as an embedded module within the image reconstruction system 103.

An approximate function generation module 105A of the surrogate calibration module 105 generates an approximate function based on the high resolution representation 104 and the captured images 101, wherein the approximate function is a function of system parameters 102. The approximate function thus generated may be represented using below equation:

dₐₚₚ(P)=d[(Simulate(S(k)_{evaluated at P}_{ᵢ},P),I_{captured}]

where dₐₚₚ represents an approximated distance metric being computed for the system parameter(s) P and Pᵢ represents system parameters from i^{th} iteration performed by method.

The approximate function thus generated is then optimized by an approximate function optimization module 105B of the surrogate calibration module 105 for generating an updated set of system parameters 102′. The approximate function optimization module 105B evaluates the approximate function at plurality of sample points in a parameter space of the system parameters 102, obtains a minimal value of the approximate function for the system parameters, and updates the system parameters corresponding to the minimal value.

The approximate function optimization module 105B for evaluating the approximate function, generates simulated images by simulating the captured images based on the high resolution representation generated in a previous iteration of calibration of system parameters, and the particular instance of the system parameters and obtains a distance metric between the simulated images and the captured images, both of which are low resolution images.

The simulated images are generated by simulating the captured images 101 based on the system parameter(s) 102 and the amplitude and phase information associated with the high resolution representation 104 using sample point(s) in a multi-dimensional parameter space of the system parameters 102. As used herein, “parameter space” refers to a range of values that a system parameter 102 may assume while capturing the captured image 101. Also, used herein “sample points” refer to the values assumable at a given time instance by two or more system parameters 102, thereby making it multi-dimensional. For example, sample points may include focus and wavelength. When two or more system parameters 102 are thus used, the calibration happens simultaneously and therefore, faster and effectively, for multiple system parameters 102 at once.

The simulated images can be represented by the equation given below:

I_{simulated}=Simulate(S(k),P)

where ‘Simulate’ represents, for example, the forward imaging function used for simulating the captured images using the system parameters. Advantageously, P that is used to generate I_{simulated} can be perturbed around the system parameters used to generate S(k), thereby allowing ample sample point evaluations at a faster rate as Simulate function is orders of magnitude faster to evaluate compared to Recon function. Thus, the optimization of the system parameters can be facilitated by optimization of the approximate function.

Upon obtaining a minimal value of the approximate function for the system parameters, the approximate function optimization module 105B updates the system parameters 102 corresponding to the minimal value, which can be represented by the equation given below:

Pᵢ₊₁=argminₚ[dₐₚₚ(P)]

Where argmin represents minimal value, Pᵢ₊₁ is the system parameter 102 at iteration ‘i+1’ that is relying on the amplitude and phase information, that is used in S(k), from the previous iteration ‘i’ of calibration of the system parameters 102. The surrogate calibration module 105 iteratively updates the system parameters 102 to form an updated set of system parameters, for example P_{i+N} where ‘N’ represents the number of iterations, which may not be more than ten. The finally updated set of system parameters 102′ is provided as an input to the image reconstruction system 103. The finally updated set of system parameters 102′ when used by the image reconstruction system 103, results in a speedy calibration of the image reconstruction system 103 which in turn enhances the quality of reconstruction of the images reconstructed by the image reconstruction system 103.

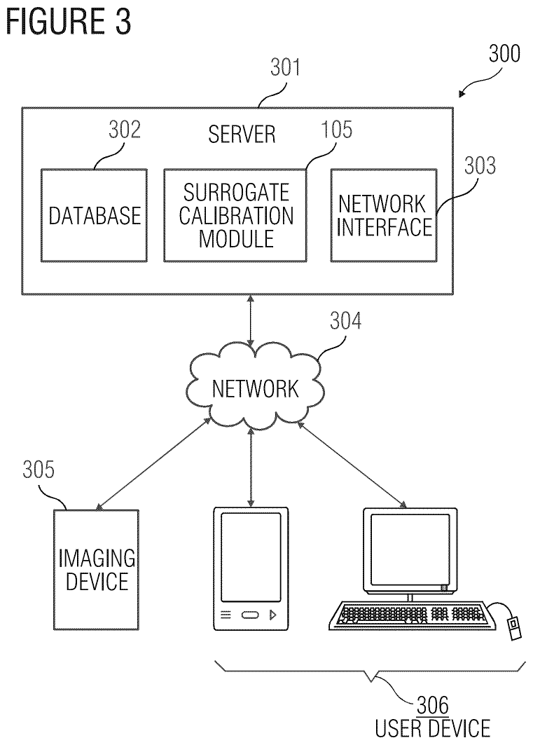

FIG. 3 provides an illustration of a block diagram of a client-server architecture that is a geometric modelling of components representing different parts of real-world objects, according to an embodiment of the present disclosure. The client-server architecture 300 includes a server 301 and a plurality of user devices 306. Each of the user device 306 is connected to the server 301 via a communication network 304, for example, local area network (LAN), wide area network (WAN), WiFi, etc. In one embodiment, the server 301 is deployed in a cloud computing environment. As used herein, “cloud computing environment” refers to a processing environment comprising configurable computing physical and logical resources, for example, networks, servers, storage, applications, services, etc., and data distributed over the communication network 304, for example, the internet. The cloud computing environment provides on-demand network access to a shared pool of the configurable computing physical and logical resources. The server 301 may include a database 302 that comprises images captured by imaging device(s) 305. The server 301 may include a surrogate calibration module 105 that is configured to calibrate the system parameters 102 in order to enhance image quality of images generated by the image reconstruction system 103. Additionally, the server 301 may include a network interface 303 for communicating with the user device 305 via the network 304.

The user devices 306 are used by users, for example, a medical personnel such as a pathologist, physician, etc. In an embodiment, the user devices 306 may be used by the user to provide captured images of the sample, receive the updated system parameters, and/or receive the enhanced images generated by the image reconstruction system 103. The data can be accessed by the user via a graphical user interface (not shown) of an end user web application on the user device 306. In another embodiment, a request may be sent to the server 301 to access the images via the network 304. An imaging device 305 may be connected to the server 301 through the network 304. The imaging device 305 may be configured to capture a plurality of images of a sample. The imaging device 305 may be, for example, a Fourier Ptychography microscope.

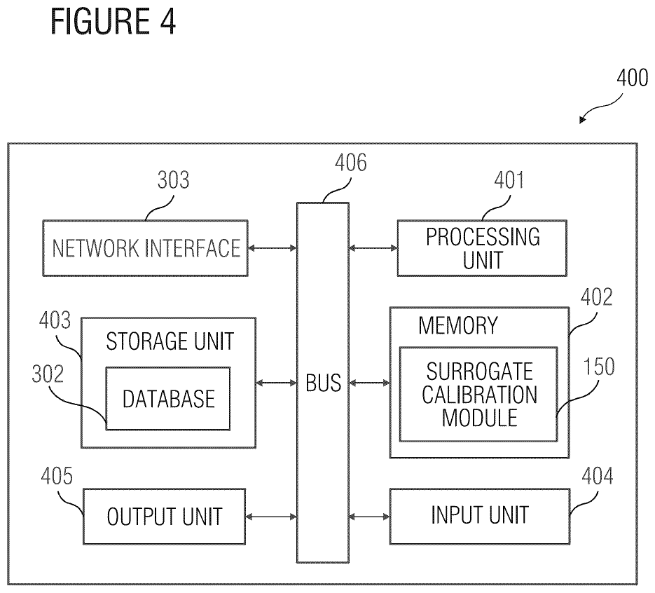

FIG. 4 is a block diagram illustrating an architecture of a computer system 400 employed by the image reconstruction system 103 shown in FIG. 2, according to an embodiment of the present disclosure. The computer system 400 comprises a processing unit 401, a memory 402, a storage unit 403, an input unit 404, an output unit 405, a bus 406, and a network interface 303.

The processing unit 401, as used herein and also referred to as the processor, means any type of computational circuit, such as, but not limited to, a microprocessor, microcontroller, complex instruction set computing microprocessor, reduced instruction set computing microprocessor, very long instruction word microprocessor, explicitly parallel instruction computing microprocessor, graphics processor, digital signal processor, or any other type of processing circuit. The processing unit 401 may also include embedded controllers, such as generic or programmable logic devices or arrays, application specific integrated circuits, single-chip computers, and the like.

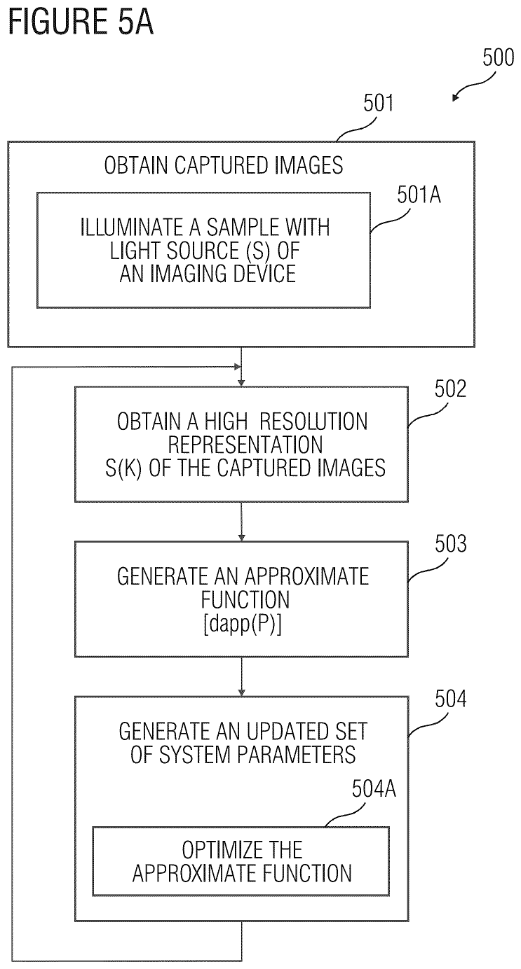

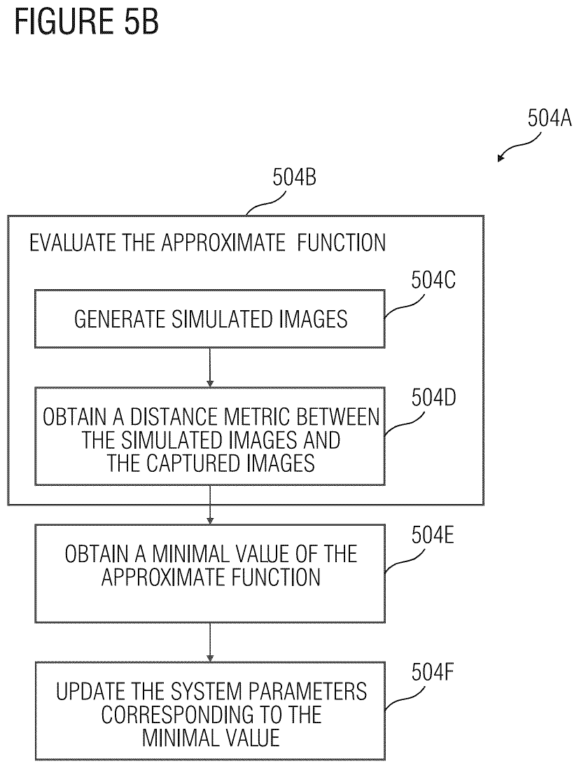

The memory 402 may be volatile memory and non-volatile memory. The memory 402 may be coupled for communication with said processing unit 401. The processing unit 401 may execute instructions and/or code stored in the memory 402. A variety of computer-readable storage media may be stored in and accessed from said memory 402. The memory 402 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 402 includes a surrogate calibration module 105 stored in the form of machine-readable instructions on any of said above-mentioned storage media and may be in communication to and executed by processor 401. When executed by the processing unit 401, the surrogate calibration module 105 causes the processing unit 401 to calibrate the system parameters associated with the image reconstruction system 103 such that the images generated or reconstructed by the image reconstruction system 103 are of enhanced image quality. Method steps executed by the processing unit 401 to achieve the abovementioned functionality are elaborated upon in detail in FIGS. 5A-5B.

The storage unit 403 may be a non-transitory storage medium which stores a database 302. The database 302 is a repository of images captured by the imaging device 305. The input unit 404 may include input means such as keypad, touch-sensitive display, camera (such as a camera receiving gesture-based inputs), etc. capable of receiving input signal such as a medical image. The bus 406 acts as interconnect between the processing unit 401, the memory 402, the storage unit 403, the input unit 404, the output unit 405 and the network interface 303.

Those of ordinary skills in the art will appreciate that said hardware depicted in FIG. 4 may vary for particular implementations. For example, other peripheral devices such as an optical disk drive and the like, Local Area Network (LAN)/Wide Area Network (WAN)/Wireless (e.g., Wi-Fi) adapter, graphics adapter, disk controller, input/output (I/O) adapter also may be used in addition or in place of the hardware depicted. Said depicted example is provided for the purpose of explanation only and is not meant to imply architectural limitations with respect to the present disclosure.

The computer system 400 in accordance with an embodiment of the present disclosure includes an operating system employing a graphical user interface. Said operating system permits multiple display windows to be presented in the graphical user interface simultaneously with each display window providing an interface to a different application or to a different instance of the same application. A cursor in said graphical user interface may be manipulated by a user through a pointing device. The position of the cursor may be changed and/or an event such as clicking a mouse button, generated to actuate a desired response. One of various commercial operating systems, such as a version of Microsoft Windows™, a product of Microsoft Corporation located in Redmond, Washington may be employed if suitably modified. Said operating system is modified or created in accordance with the present disclosure as described. Disclosed embodiments provide systems and methods for processing images and particularly medical images. In particular, the systems and methods are directed towards enhancing image quality of images.

Disclosed herein is also a computer program product comprising a non-transitory computer readable storage medium that stores computer program codes comprising instructions executable by at least one processing unit 401 for calibrating system parameters 102 associated with an image reconstruction system 103. The computer program product comprises a first computer program code for obtaining captured images 101 by illuminating a sample with one or more light sources associated with an imaging device 305 of the image reconstruction system 103; a second computer program code for obtaining a high resolution representation 104 of the captured images 101, wherein the high resolution representation 104 is generated by the image reconstruction system 103 based on the system parameters 102 and the captured images 101; a third computer program code for generating an approximate function based on the high resolution representation 104 and the captured images 101, wherein the approximate function is a function of the system parameters 102; and a fourth computer program code for generating an updated set of system parameters by optimizing the approximate function.

In an embodiment, a single piece of computer program code comprising computer executable instructions, performs one or more steps of the computer implemented method according to the present disclosure, for calibrating system parameters 102 associated with an image reconstruction system 103. The computer program codes comprising computer executable instructions are embodied on the non-transitory computer readable storage medium. The processing unit 401 of the computer system 400 retrieves these computer executable instructions and executes them. When the computer executable instructions are executed by the processing unit 401, the computer executable instructions cause the processing unit 401 to perform the steps of the method for calibrating system parameters 102 associated with an image reconstruction system 103.

FIGS. 5A-5B illustrate flowcharts of a computer-implemented method 500 for calibrating system parameters 102 associated with the image reconstruction system 103 shown in FIG. 2, according to an embodiment of the disclosure.

As shown in FIG. 5A, at step 501, the computer-implemented method obtains captured images 101. For obtaining the captured images, at step 501A, the method illuminates a sample with multiple light sources of an imaging device 305 of the image reconstruction system 103. The captured images 101 are low resolution image(s). According to an embodiment, the imaging device 305 is a Fourier Ptychography microscope. The imaging device 305 may include an imaging module comprising one or more light sources such as light emitting diodes (LEDs), a tube lens, one or more objective lenses and an image capturing unit. The light sources may have specific wavelength distribution. In an embodiment, the light emitted from the light sources passes through a microscopic slide which includes the sample to be imaged. The objective lens assembly in the imaging module may be used to visualize and magnify the one or more components on the microscopic slide. The tube lens is used in microscopes to enable creation of real images from intermediate images placed at infinity. Therefore, tube lens enables visualization of infinity corrected images. The image capturing unit may include imaging lenses and an imaging sensor, configured to capture an image of the illuminated microscopic slide. The imaging sensor may be, for example a charge-coupled device (CCD) or a complementary metal oxide semiconductor (CMOS). In an embodiment, the light source includes multiple LEDs. Each LED may be configured to emit light at a predefined angle on to the sample. An image may be obtained/captured for each of the predefined angles and may be stitched to obtain a final image of the sample.

At step 502, the method obtains a high resolution representation 104 of the captured images 101. The high resolution representation 104 is generated by the image reconstruction system 103 based on the captured images 101 and the system parameters 102, for example, an initial guess of the system parameters 102. The system parameters 102 include, for example, optical data, geometrical data, alignment data, illumination plane to sample separation data, magnification data, wavelength data, and/or thresholding data. Advantageously, it is assumed that the physical state of the imaging device 305 remains nearly constant throughout the calibration. The system parameters 102 are obtained, for example, fetched from a parameter database (not shown) that stores nominal system parameters or system parameters from previous calibrations of the image reconstruction system 103. The frequency of calibration of the system parameters 102, for example, weekly, monthly, etc., may be defined based on the stability and the variability of the image reconstruction system 103. The high resolution representation 104 can be represented by the equation given below:

S(k)=Recon(I_{captured},P)

where S(k) is the high resolution representation 104, ‘Recon’ represent the reconstruction function, I_{captured} represents the captured images 101 of low resolution and P represents the system parameters 102.

At step 503 the method generates an approximate function based on the high resolution representation 104 and the captured images 101. The approximate function is a function of the system parameters 102. The approximate function thus generated may be represented using below equation:

dₐₚₚ(P)=d[(Simulate(S(k)_{evaluated at P}_{ᵢ},P),I_{captured}]

where dₐₚₚ represents an approximated distance metric being computed for the system parameter(s) P and Pᵢ represents system parameters from i^{th} iteration performed by method.

At step 504, the method generates an updated set of system parameters 102′. At step 504A, the method optimizes the approximate function to generate the updated set of system parameters 102′. The steps 502-504 are performed iteratively by the method disclosed herein for generating the updated set of system parameters 102′ in a faster and an efficient manner which in turn enhances the quality of reconstruction of the images reconstructed by the image reconstruction system 103. According to an embodiment, the number of iterations are predefined based on the heuristics. According to another embodiment, the number of iterations are a function of the high resolution representation 104 such that the high resolution representation comprises sufficient resolution and minimal noise and artifacts.

As shown in FIG. 5B, the method for optimizing the approximate function, at step 504B, evaluates the approximate function at multiple sample points in a parameter space of the system parameters 102. For evaluating the approximate function, the method, at step 504C, generates simulated images by simulating the captured images 101 based on the high resolution representation 104 generated in a previous iteration of calibration of system parameters 102 as shown in Figured 5A, and the particular instance of the system parameters 102. The simulated images are generated by simulating the captured images 101 based on the system parameters 102 and the amplitude and phase information of the high resolution representations 104. The simulated images, for example, may be generated using a forward model by simulating the captured images using sample points, for example, in a multi-dimensional parameter space associated with the system parameters 102 and the amplitude and phase information of the high resolution representation 104. The simulated image may be represented by the below equation:

I_{simulated}=Simulate(S(k),P)

where ‘Simulate’ represents, for example, the forward imaging function used for simulating the captured images using the system parameters. Advantageously, P that is used to generate I_{simulated} can be perturbed around the system parameters 102 used to generate S(k), thereby allowing ample sample point evaluations at a faster rate as Simulate function is orders of magnitude faster to evaluate compared to Recon function. Thus, the optimization of the system parameters can be facilitated by optimization of the approximate function.

For evaluating the approximate function, the method, at step 504D, obtains a distance metric between the simulated images and the captured images 101, both of which are low resolution images. The distance metric, that is an approximated distance metric, between the simulated images and the captured images 101, according to one embodiment, is obtained at pixel level, for example, by representing each of the images into a matrix of column and row vectors.

The method for optimizing the approximate function, at step 504E, obtains a minimal value of the approximate function for the system parameters 102 and then at step 504F, updates the system parameters 102 corresponding to the minimal value. For obtaining a minimal value of the approximate function, a minimal value of the distance metric is obtained for example if the distance metric is treated as a function corresponding to the system parameters 102, attaining different values of sample points based on the difference between the simulated images and the captured images 101, then an absolute minimum value of this function is obtained.

Upon obtaining a minimal value of the approximate function for the system parameters 102, the method updates the system parameters 102 corresponding to the minimal value, which can be represented by the equation given below:

Pᵢ₊₁=argminₚ[dₐₚₚ(P)]

Where argmin represents minimal value, Pᵢ₊₁ is the system parameter 102 at iteration ‘i+1’ that is relying on the amplitude and phase information, that is used in S(k), from the previous iteration ‘i’ of calibration of the system parameters 102, that is, the steps 502-504 as shown in FIG. 5A. The finally updated set of system parameters 102′ is provided as an input to the image reconstruction system 103. The finally updated set of system parameters 102′ when used by the image reconstruction system 103, results in a speedy calibration of the image reconstruction system 103 which in turn enhances the quality of reconstruction of the images reconstructed by the image reconstruction system 103. Advantageously, approximating the distance metric significantly reduces the number of iterations that involves image reconstruction steps compared to the optimization techniques used for calibration as per state of the art.

Advantageously, the present disclosure enables removal of artifacts and reconstruction noise in images generated using Fourier Ptychography microscope. The computer-implemented method, system, device and the computer program product disclosed herein enable an improved reconstructed image of the sample in lieu of efficiently and finely calibrated system parameters 102.

The foregoing examples have been provided merely for the purpose of explanation and are in no way to be construed as limiting of the present disclosure disclosed herein. While the disclosure has been described with reference to various embodiments, it is understood that the words, which have been used herein, are words of description and illustration, rather than words of limitation. Further, although the disclosure has been described herein with reference to particular means, materials, and embodiments, the disclosure is not intended to be limited to the particulars disclosed herein; rather, the disclosure extends to all functionally equivalent structures, methods and uses, such as are within the scope of the appended claims. Those skilled in the art, having the benefit of the teachings of this specification, may affect numerous modifications thereto and changes may be made without departing from the scope and spirit of the disclosure in its aspects.

What is claimed is:

1. A computer implemented method for calibrating system parameters associated with an image reconstruction system; the method comprising:
obtaining captured images by illuminating a sample with one or more light sources associated with an imaging device of the image reconstruction system;
characterized by iteratively performing:
obtaining a high resolution representation of the captured images, wherein the high resolution representation is generated by the image reconstruction system based on the system parameters and the captured images;
generating an approximate function based on the high resolution representation and the captured images, wherein the approximate function is a function of the system parameters; and
generating an updated set of system parameters by optimizing the approximate function.

2. The method according to claim 1, wherein the system parameters comprise one or more of optical data, geometrical data, alignment data, illumination plane to sample separation data, magnification data, wavelength data, and thresholding data.

3. The method according to claim 1, wherein optimizing the approximate function comprises:
evaluating the approximate function at plurality of sample points in a parameter space of the system parameters;
obtaining a minimal value of the approximate function for the system parameters; and
updating the system parameters corresponding to the minimal value.

4. The method according to claim 3, wherein evaluating the approximate function at a particular instance of the system parameters, comprises:
generating simulated images by simulating the captured images based on the high resolution representation generated in a previous iteration of calibration of system parameters, and the particular instance of the system parameters; and
obtaining a distance metric between the simulated images and the captured images.

5. The method according to claim 4, wherein the captured images and the simulated images are low resolution images.

6. An imaging device of an image reconstruction system for calibrating system parameters associated with an image reconstruction system, comprising:
an imaging module configured to illuminate a sample with one or more light sources of the imaging device and capture plurality of images of the sample, wherein the captured images are low resolution images;
one or more processing units; and
a memory coupled to the one or more processing units, the memory comprising a surrogate calibration module configured to iteratively:
obtain a high resolution representation of the captured images, wherein the high resolution representation is generated by the image reconstruction system based on the system parameters and the captured images, wherein the system parameters comprise one or more of optical data, geometrical data, alignment data, illumination plane to sample separation data, magnification data, wavelength data, and thresholding data;
generate an approximate function based on the high resolution representation and the captured images, wherein the approximate function is a function of the system parameters; and
generate an updated set of system parameters by optimizing the approximate function.

7. The imaging device according to claim 6, wherein optimizing the approximate function comprises:
evaluating the approximate function at plurality of sample points in a parameter space of the system parameters;
obtaining a minimal value of the approximate function for the system parameters; and
updating the system parameters corresponding to the minimal value.

8. The imaging device according to claim 7, wherein evaluating the approximate function at a particular instance of the system parameters, comprises:
generating simulated images by simulating the captured images based on the high resolution representation generated in a previous iteration of calibration of system parameters, and the particular instance of the system parameters; and
obtaining a distance metric between the simulated images and the captured images, wherein the captured images and the simulated images are low resolution images.

9. The imaging device according to claim 6, further comprising:
one or more controllable light sources placed at a discrete position;
a tube lens;
one or more objective lens; and
an imaging capturing unit.

10. An image reconstruction system for calibrating system parameters, comprising:
one or more servers;
an imaging device coupled to the one or more servers;
the one or more servers comprising instructions, which when executed causes the one or more servers to:
obtain captured images by illuminating a sample with one or more light sources associated with an imaging device of the image reconstruction system; and
iteratively perform:
obtaining a high resolution representation of the captured images, wherein the high resolution representation is generated by the image reconstruction system based on the system parameters and the captured images, wherein the system parameters comprise one or more of optical data, geometrical data, alignment data, illumination plane to sample separation data, magnification data, wavelength data, and thresholding data;
generating an approximate function based on the high resolution representation and the captured images, wherein the approximate function is a function of the system parameters; and
generating an updated set of system parameters by optimizing the approximate function.

11. The image reconstruction system according to claim 10, wherein in optimizing the approximate function, the one or more servers perform below steps:
evaluating the approximate function at plurality of sample points in a parameter space of the system parameters;
obtaining a minimal value of the approximate function for the system parameters; and
updating the system parameters corresponding to the minimal value.

12. The image reconstruction system according to claim 11, wherein in evaluating the approximate function at a particular instance of the system parameters, the one or more servers perform below steps:
generating simulated images by simulating the captured images based on the high resolution representation generated in a previous iteration of calibration of system parameters, and the particular instance of the system parameters; and
obtaining a distance metric between the simulated images and the captured images, wherein the captured images and the simulated images are low resolution images.

13. A computer-program product for calibrating system parameters associated with an image reconstruction system, having machine-readable instructions stored therein, that when executed by one or more processing units, causes the processing units to:
obtain captured images by illuminating a sample with one or more light sources associated with an imaging device of the image reconstruction system; and
iteratively perform:
obtaining a high resolution representation of the captured images, wherein the high resolution representation is generated by the image reconstruction system based on the system parameters and the captured images, wherein the system parameters comprise one or more of optical data, geometrical data, alignment data, illumination plane to sample separation data, magnification data, wavelength data, and thresholding data;
generating an approximate function based on the high resolution representation and the captured images, wherein the approximate function is a function of the system parameters; and
generating an updated set of system parameters by optimizing the approximate function.

14. The computer-program product according to claim 13, wherein optimizing the approximate function, comprises:
evaluating the approximate function at plurality of sample points in a parameter space of the system parameters; and
obtaining a minimal value of the approximate function for the system parameters; and
updating the system parameters corresponding to the minimal value.

15. The computer-program product according to claim 14, wherein evaluating the approximate function at a particular instance of the system parameters, comprises:
generating simulated images by simulating the captured images based on the high resolution representation generated in a previous iteration of calibration of system parameters, and the particular instance of the system parameters; and
obtaining a distance metric between the simulated images and the captured images, wherein the captured images and the simulated images are low resolution images.
